(19) United States
(12) Patent Application Publication
(10) Pub. No.: US 2024/0141284 A1
(43) Pub. Date: May 2, 2024

(54) COMPOSITIONS AND METHODS FOR CULTIVATING ENVIRONMENTAL MICROORGANISMS AND COMPOSITIONS DERIVED THEREFROM

(71) Applicant: Bien-Etre Labs, LLC, High Point, NC (US)

(72) Inventors: Adnan Mjalli, High Point, NC (US); Chitra Wendakoon, High Point, NC (US)

(21) Appl. No.: 18/279,623

(22) PCT Filed: Mar. 3, 2022
(86) PCT No.: PCT/US2022/070946

§ 371 Date: Aug. 31, 2023

Related U.S. Application Data
(60) Provisional application No. 63/156,011, filed on Mar. 3, 2021.

(51) Int. Cl. C12N 1/20; C12Q 1/04

(52) U.S. Cl. CPC C12N 1/20; C12Q 1/04

(57) ABSTRACT

Disclosed are compositions and methods for cultivating environmental microorganisms using plant-based media. Also disclosed herein are methods of characterizing environmental samples by culturing the environmental sample, identifying microorganisms and determining the biological activity of the microorganisms.

### RELATED APPLICATIONS

This application claims priority to U.S. Provisional Application No. 63/156,011 filed Mar. 3, 2021. The disclosure of U.S. Provisional Application No. 63/156,011 is incorporated by reference in its entirety herein.

### FIELD

This present disclosure relates to compositions and methods for cultivating environmental microorganisms and compositions derived therefrom

### BACKGROUND

Microorganisms present in the environment are highly diverse. Only a tiny fraction of soil microbiota is readily culturable with conventional growth media. One of the challenges in culturing environmental bacteria is the requirement for highly specific nutritional compounds to facilitate the growth of diverse organisms present in the environment. Developing compositions and methods to cultivate the enormously diverse microflora including fastidious organisms, can be a difficult task requiring more specific, improved media. Several commercially available media such as brain heart infusion (BHI) medium, tryptic soy broth (TSB) medium and media supplemented with additional vitamins and salts have been widely used for the cultivation of environmental bacteria. These media are primarily made with animal-derived components, which increases the cost of the media. Given these challenges, equally effective, but inexpensive culture media are necessary for cultivation of complex microbial consortia.

The rich diversity of organisms such as bacteria, fungi, viruses and algae form interactive and complex microbial communities that present a vast range of opportunities to explore their specific functions. For example, soil microorganisms have a direct influence on various ecosystem-associated processes performing critical functions in nutrient cycles, such as the nitrogen and carbon cycles, and degradation of soil contaminants (Johns, C. (2017) Living soils: the role of microorganisms in soil health. Future Directions International, HTTP://futuredirections.org.au). Understanding microbial activities such as metabolizing and releasing macronutrients for plant growth, repelling pathogenic microbial strains, stimulating plant growth via signaling hormones, and degrading soil contaminants, aids in the development of novel applications related to microbial functions. The use of soil microorganisms as alternative agents that improve environment quality such as in biodegradation and bio-stimulation have been recognized as sustainable processes for restoration of impacted soil (M Vidali (2001) Bioremediation. An overview. Pure and Applied Chemistry 73:7, 1163-1172).

Such methods and compositions allow for studies to characterize environmental samples and would contribute to the identification and isolation of microorganisms with particular biological activities. Thus, facilitating the development of novel formulations for agricultural and biotechnological purposes.

### SUMMARY

It is an object of the present disclosure to provide compositions and methods for cultivating environmental microorganisms and compositions derived therefrom. The compositions and methods may be embodied in a variety of ways.

In some embodiments, the present disclosure provides a method of using a plant-based growth medium to simultaneously cultivate a consortium of distinct (i.e., different strains) microorganisms, wherein the consortium is at least 2 distinct microorganisms, optionally at least 5 distinct microorganisms, optionally at least 10 distinct microorganisms, optionally at least 20 distinct microorganisms, or optionally at least 50 distinct microorganisms in a sample. In certain embodiments, the growth medium comprises a portion of a date palm fruit.

In other embodiments, the present disclosure provides a method of characterizing an environmental sample comprising: (i) obtaining an environmental sample; (ii) culturing the sample using a plant-based growth medium; (iii) identifying at least one microorganism in the sample; and (iv) optionally determining the biological activity of at least one microorganism identified in step (iii).

In other embodiments, the present disclosure provides a composition comprising at consortium of the microorganisms identified and optionally characterized as disclosed herein, wherein the consortium is at least 2 distinct microorganisms, optionally at least 5 distinct microorganisms, optionally at least 10 distinct microorganisms, optionally at least 20 distinct microorganisms, or optionally at least 50 distinct microorganisms.

### BRIEF DESCRIPTION OF THE DRAWINGS

The present invention may be better understood by referring to the following non-limiting figures.

FIG. 1 shows the growth of Gaia Flush bacteria in various types of media. The date palm media with supplementation showed equal growth (approximately log 10⁷ CFU/mL) to the commercially available media.

FIG. 2 depicts a cross streak method for antibacterial activity of two Bacillus subtilis strains against Staphylococcus aureus ATCC 25923. Bacillus subtilis strains were streaked vertically on the agar perpendicular to the horizontal streaks of Staphylococcus aureus ATCC 25923. The clear zones demonstrate the antibacterial activity of the Bacillus subtilis strains.

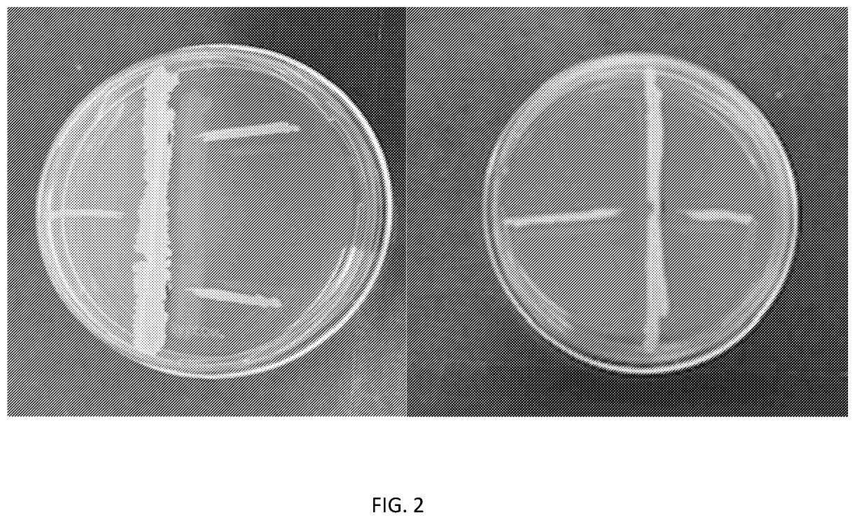

### DETAILED DESCRIPTION

The following description recites various aspects and embodiments of the present compositions and methods. No particular embodiment is intended to define the scope of the compositions and methods. Rather, the embodiments merely provide non-limiting examples of various methods and systems that are at least included within the scope of the compositions and methods. The description is to be read from the perspective of one of ordinary skill in the art; therefore, information well known to the skilled artisan is not necessarily included.

### Definitions

The present disclosure now will be described more fully hereinafter. The disclosure may be embodied in many different forms and should not be construed as limited to the aspects set forth herein; rather, these aspects are provided so that this disclosure will satisfy applicable legal requirements. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

When introducing elements of the present disclosure or the embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements. It is understood that aspects and embodiments of the disclosure described herein include “consisting” and/or “consisting essentially of” aspects and embodiments.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B, and/or C” is intended to mean A alone, B alone, C alone, A and B in combination, A and C in combination, B and C in combination, or A, B, and C in combination.

Various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

“Sample” or “environmental sample” are used interchangeably herein. The source of the sample may be from air, water, soil, biological materials, and waste. Environmental samples may be liquids, aerosols, solids, or sludges. Additionally, environmental samples may be formulations comprising environmental microorganisms. For example, Gaia Flush (Gaia Klēn, LLC) is a molasses-based formulation comprising unidentified soil microorganisms.

As used herein, the term “date palm medium” or “date medium” means a microorganism growth media comprising a portion of a date palm fruit. The portion of the date palm fruit may be from the flesh and/or pit of the fruit. Date broth can be prepared by soaking date paste in water or from mixing dried date powder with water. For example, in some embodiments, date broth is prepared by soaking date 150 g of date paste in distilled water and heated at 60° C. for 3 h while mixing every 30 min. The resulting date slurry may then be mixed for another 1 h at room temperature and filtered through a cheesecloth to obtain the date broth. In another embodiment, 150 g of dried date powder may be combined with 1 L of distilled water and filtered through cheesecloth to obtain the date broth. In some embodiments, the date broth is the only source on nutrients in the growth medium.

As used herein, a “distinct microorganism” means a microorganism that is different from another microorganism in the sample. In various embodiments, the microorganism may be a distinct species, characterized as having a unique (i.e., <97% sequence identity) in the variable region of the 16S rRNA sequence or portion thereof as disclosed herein. In other embodiments, the unique microorganism may be a distinct strain of the same species having greater than or equal to 97% identity with the variable region of the 16S rRNA but less than 100% identity.

The term “formulation” means a consortium of beneficial living microorganisms dispersed or trapped in solid or liquid carriers allowing effective delivery.

The term “16S rRNA gene sequencing” refers to sequence based bacterial analysis. The 16S ribosomal (r) RNA gene of bacteria encodes the small subunit of ribosomal RNA. The RNA sequences have been used extensively in the classification and identification of bacteria. The sequence of the 16S rRNA gene contains two regions: conserved and variable regions. The highly variable regions reflect differences between species. The comparison of the complete 16S rRNA gene sequence of an isolate against other sequences of close strains provides a rapid and accurate method to identify the bacterial strains. This method has been widely used to identify species by establishing taxonomic relationship between the strains with 98% similarity as the cutoff for distinguishing species (Johnson et al. (2019) Evaluation of 16S rRNA gene sequencing for species and strain-level microbiome analysis. Nature Communications, 10, 5029).

The terms “percent identity” or “percent of identity” refer to a quantitative measurement of the similarity between two sequences. By comparing two locally aligned sequences using an established 16S rRNA sequence database, the percent of sequence identity can be determined. The NCBI BLAST program, which is available at http://blast.ncbi.nih.gov is one of the databases used to determine the percentage of alignment between sequences.

### Method of Cultivating Microorganisms

In some embodiments, the present disclosure provides a method for using a plant-based growth medium to simultaneously cultivate a consortium of microorganisms. In some embodiments, the consortium of microorganisms is at least 2, 5, 10, 20, 30, 40, 50, 60, 70, 80, 90, or 100 distinct (i.e., different) strains of microorganisms. In some embodiments, the consortium of microorganisms is at least 2, 5, 10, 20, 30, 40, 50, 60, 70, 80, 90, or 100 distinct (i.e., different) species of microorganisms.

In certain embodiments the plant provides nutrients sufficient for growth of the consortium of microorganisms. Microorganisms have specific nutritional requirements for growth. These nutritional requirements may vary widely depending on the genera, species, and strain of the microorganisms. Generally, microorganisms require carbohydrates, nitrogen, amino acids, vitamins, and minerals for growth. In some embodiments, a plant or a portion of a plant may be the source of nutrients in the growth medium.

In certain embodiments, the plant-based medium comprises at least a portion of a vegetable or a fruit. In some instances, the fruit is a date palm. Date palm fruits are a rich source of sugars, vitamins, minerals, and dietary fiber (Mohamed, R M A, Fageer, A S M, Mohamed M., Ahmed E I A M, (2014) Food Science & Nutrition 2014; 2(5): 478-489), and can be a good growth medium for cultivating any type of microorganisms. In certain embodiments, the use of date palm fruit, as an alternative to standard conventional media (e.g., BHI, TSA, and MRS), might be advantageous as providing glucose and fructose as well as minerals present in these fruits. Additionally, date palm fruit is readily available, thereby lowering the cost of the culture media. In certain embodiments, the date palm medium may contain supplements as disclosed herein.

The fruit of the date palm (Phoenix dactylifera L.) is one of the most widely grown fruits in the world, typically, planted in hot dry regions. However, large amounts of dates end up as byproducts or waste. Date fruit progress through four stages of maturation: (i) Hababouk (ii) Kimiri; (iii) Khalal; (iv) Rutab; and (v) Tamar. Dates are primarily composed of water, sugar, protein, fat, pectin, minerals, vitamins, crude fiber, antioxidants, and polyphenols. As dates mature, the moisture and sugar contents change. For example, unripe Khalal dates are 50% moisture while, mature Tamar dates have 10-30% moisture content. In some embodiments, the date fruit is a Tamar date. Tamar dates have reached commercial maturity and the fruit has lost significant amounts of water, which prevents fermentation and ensures conservation of the fruit. Tamar dates are used commercially to produce a variety of date products (e.g., juice, syrup, jam, and jelly). In some embodiments, the date fruit is a commercial byproduct of a Tamar date processing.

Dates are a rich source of carbohydrates (mainly starch and sugars), some amino acids, vitamins (vitamin A, vitamin C, thiamin (B1), riboflavin (B2), niacin, and vitamin E), minerals (calcium, iron, magnesium, phosphorus, potassium, sodium, and zinc), dietary fiber, and also contain other minor nutrients such as antioxidants, triglycerides, linoleic acid, and palmitic acid. However, the date fruit industry produces a large amount of waste due to unwanted byproducts. Each year, more than 55,000 tons of dates are wasted due to low quality and/or unusable byproducts. The nutritional compositions of date byproducts allow them to be utilized to develop growth media for cultivating a variety of microorganisms.

Differing date cultivars are also characterized by varying levels of moisture, protein, glucose, fructose, sucrose, and fiber. In certain embodiments, a specific cultivar of dates is used. The date cultivar may be Barhi, Deglet Noor, Fard. Hallawi, Khardraqy, Khalas, Khasab, Lulu, Madjool, or Zahidi, or any combination thereof. In some embodiments, multiple date cultivars may be used. Khalas dates are typically composed of about 36% glucose, about 32% fructose and <0.05% sucrose.

Dates typically contain small amounts of disaccharides (lactose, maltose, and sucrose). As dates develop, the sucrose content decreases as the sucrose is converted into monosaccharide sugars (fructose and glucose). In some instances, the provided methods produce date palm broth comprising <0.05% maltose, <0.05% lactose and/or <0.05% sucrose. In some embodiments, the total sugar content is <25%, <20%, <15%, or <10%. For example, in one embodiment, date palm medium comprises about 14.37% total sugar content.

Dates contain both soluble and insoluble fibers. In certain embodiments, dates of a defined fiber content and/or composition are used. For example, in certain embodiments, the main component is cellulose, hemicelluloses, pectin, and lignin. The lignin composition is divided into low- and high-lignin separated from palm date fibers. High-lignin fibers contain 75% lignin and 15.8% polysaccharide, while the low-lignin fibers contain 27.2% lignin and 53.1% polysaccharides. Palm date fiber consists of 54% high-lignin fiber and 46% low-lignin fiber. In dates, insoluble fiber is the major component of dietary fiber; on the other hand, soluble pectin continues to accumulate regularly until the date fruit reaches the Rutab (ripened) stage.

The nutritional components of dates be In some embodiments the growth medium comprises date palm paste. In some embodiments, the medium comprises date powder. Various date palm pastes and powders are commercially available. The nutritional composition of two commercially available (Al Ameen and LIWA) date palm pastes and date palm powders are provided in Table 1.

In some embodiments, a date broth is prepared from date palm paste or date palm powder. Date broth can be prepared by soaking date paste in water or from mixing dried date powder with water. For example, in some embodiments, date broth is prepared by soaking date 150 g of date paste in distilled water and heated at 60° C. for 3 h while mixing every 30 min. The resulting date slurry may then be mixed for another 1 h at room temperature and filtered through a cheesecloth to obtain the date broth. In another embodiment, 150 g of dried date powder may be combined with 1 L of distilled water and filtered through cheesecloth to obtain the date broth. In some embodiments, the date broth is the only source on nutrients in the growth medium.

In some embodiments, the growth medium is supplemented with additional nutrients required for microorganism growth. For normal growth, culture media are supplemented with various free amino acids, peptides, nucleic acid derivatives, fatty acid esters, minerals, vitamins, and buffering agents in addition to sugar. Nitrogen sources such as soy peptones, peptones of animal origin, bactopeptone, tryptose, and beef extract or yeast extract form an essential part of growth media. These nitrogen sources are rich in diversified free amino acids, peptides, and most of the required vitamins and minerals, but they are expensive and thus constitute a significant portion of media costs. For example, commonly used media, e.g., DeMan Rogosa Sharpe (MRS) media, typically contains glucose, yeast and meat extracts, casein peptones, and salts and is therefore expensive to produce. Consequently, a low cost alternative medium would be beneficial. Replacing expensive nitrogen sources with lower cost ingredients such as food or agricultural byproducts could prove useful.

Calcium chloride is an important growth factor for some strains of bacteria, enhancing several enzymatic activities and aiding in cell division. In some embodiments, the concentration of calcium chloride in the supplement solution is at least 0.20 g/L, at least 0.25 g/L, or at least 0.30 g/L.

In some embodiments, the supplement solution comprises a nitrogen source of non-animal origin. The nitrogen source in a medium for cultivating microorganisms is typically meat. However, animal products can be expensive and are difficult to procure. Thus, in some embodiments, peptones of non-animal origin are used as a source of nitrogen. Peptones are the product of the hydrolysis process of protein materials and are a source of nitrogen, carbon, minerals, and growth factors. The peptone composition may vary depending on the starting protein material, enzymes, and method for hydrolysis. The starting protein can be of animal or non-animal origin. Peptones of animal origin may include meat peptones (e.g., bactopeptone, beef extract, tryptose, and proteose). Animal-free peptones may include soy peptones and yeast extracts. In some embodiments, the present invention utilizes peptones of non-animal origin as a nitrogen source. Alternatively, the nitrogen source may be any suitable source of nitrogen for culturing the bacteria of interest.

In some embodiments, the nitrogen source is at least one of a peptone, tryptone, proteose peptone, phytone peptone, polypeptone peptonetryptic soy broth, or yeast extract. Peptones are the hydrolyzed protein product from enzymatic or acidic digestion. For example, in some embodiments, the nitrogen source is Phytone™ peptone, a papiac digest of soybean meal with high vitamin and high carbohydrate content. In another example, the nitrogen source is Yeast Extract, an autolysate of harvested yeast, saccharomyces spp.

One or more surfactants may, in certain embodiments, be added in combination to formulations of the buffer solution. Surfactants use may include, but are not limited to polyoxyethylene sorbitan alkyl esters, and/or Polysorbates 20, 21, 40, 60, 65, 80, 81, and 85. For example, the surfactant used may be TWEEN® 20 and/or 80. In certain embodiments, the concentration of the surfactant in the buffer solution may range from about 2.0-3.5% (v/v) depending on the bacterial strain used, from about 2.2-3.0% (v/v), or from about 2.3-2.5% (v/v).

In some embodiments the growth medium is supplemented with one or more of the following: L-cysteine hydrochloride, di-sodium phosphate, ammonium citrate, sodium acetate, calcium chloride, potassium phosphate, magnesium sulfate, manganese sulphate, arginine, Tween-80, and yeast extract. In some embodiments, the supplement solution may comprise L-Cysteine, HCl (1.5 g), disodium phosphate (2 g), ammonium citrate (2 g), sodium acetate (5 g), calcium chloride (0.3 g), MgSO₄·7H₂O (0.2 g), MnSO₄·5H₂O (0.05 g), arginine (0.5 g), yeast extract (20 g) and TWEEN 80 (3 mL) per 1 L of solution. In certain instances the date palm medium is an acidified medium. In some embodiments, the date palm medium comprises acetic acid. In some embodiments, the date palm medium comprises a 3:2 ratio of date broth:supplement solution.

In some embodiments, the sample is an environmental sample. For example, environmental samples include, but are not limited to samples taken from air, water, soil, biological materials, and waste. Environmental samples may be liquids, aerosols, solids, or sludges. In some embodiments, the environmental sample is a known formulation. For example, in certain embodiments, Gaia Flush is a soil-based formulation product comprising several genera of previously unidentified microorganisms. In certain instances the sample comprises at least 5, 10, 20, 30, 40, 50, 60, 70, 80, 90, or 100 strains of microorganisms.

Conditions such as temperature, pH, oxygen and growth media nutritional content can affect bacteria growth activity. In some embodiments, microorganisms are grown in anaerobic environments. pH ranges desirable for bacterial growth varies by species. In some embodiments, the pH ranges from 4 to 9 depending on the species of bacteria being cultivated. In some embodiments, the microorganisms are cultured at a temperature ranginff from about 30° C. or 37° C. In other embodiments, bacteria are cultivated under microaerophilic conditions. Microaerophilic conditions provide oxygen, but at levels lower than those present in the atmosphere.

### Method of Characterizing an Environmental Sample

In another aspect, disclosed herein is a method of characterizing an environmental sample comprising (i) obtaining an environmental sample; (ii) culturing the sample using a plant-based growth medium; (iii) identifying at least one microorganism in the sample; and (iv) optionally determining the biological activity of at least one microorganism identified in step (iii). Characterization of the unknown microorganisms can be useful in development of novel microorganism formulations. In certain embodiments, the novel microorganism formulations may have various health (e.g., human health) and/or environmental uses.

In some embodiments, the sample is an environmental sample. For example, environmental samples include, but are not limited to samples may be taken from air, water, soil, biological materials, and waste. Environmental samples may be liquids, aerosols, solids, or sludges. In some embodiments, the environmental sample is a known formulation In some embodiments, the methods described herein may be used to characterize the composition of a microorganism formulation comprising unidentified microorganisms. For example, Gaia Flush (Gaia Klēn, LLC), a molasses-based formulation, is a formulation comprising unidentified soil microorganisms used for bioremediation and wastewater treatment. Gaia Flush comprises a consortium of different genera of microorganisms; however, the composition is unknown. Characterizing the environmental microorganisms and determining the specific functional properties/biological activity of individual microorganisms or a consortium of microorganisms can enable the development of novel products for bioremediation or biopharmaceutical purposes.

In certain instances, the sample comprise at least 2, 3, 5, 10, 20, 30, 40, 50, 60, 70, 80, 90, or 100 distinct strains of microorganisms. In some instances, the sample comprise at least 2, 3, 5, 10, 20, 30, 40, 50, 60, 70, 80, 90, or 100 distinct species of microorganisms.

Environmental microorganism communities consist of a wide range of microorganisms, including both fast-growing and slow-growing species. Some rapidly growing microorganisms can outgrow slow-growing species and the detection of microorganisms still presents a challenge. In some embodiments, the environmental sample is cultured for a prolonged period of time to obtain pure isolates of additional slow-growing microorganisms. In some embodiments, the microorganisms are cultured for at least 1, 2, 3, 4, 5, 6, 7, 8, 9, or 10 days. Thus, in certain embodiments, the compositions provided herein are used to cultivate microorganisms having a wide-range (i.e., slow growing vs. fast growing; specific nutrient requirements) of growth requirements.

Conditions such as temperature, pH, oxygen and growth media nutritional content can affect bacteria growth activity. In some embodiments, microorganisms are grown in anaerobic environments. pH ranges desirable for bacterial growth varies by species. In some embodiments, the pH ranges from 4 to 9 depending on the species of bacteria being cultivated. In some embodiments, the microorganisms are cultured at a temperature ranginff from about 30° C. or 37° C. In other embodiments, bacteria are cultivated under microaerophilic conditions. Microaerophilic conditions provide oxygen, but at levels lower than those present in the atmosphere.

In some embodiments, the sample is cultured using a plant-based growth medium. In certain embodiments, the plant-based medium comprises at least a portion of a vegetable or a fruit. In some instances, the fruit is a date palm. The portion of the date palm fruit may be from the flesh and/or pit of the fruit. In certain embodiments, the growth media comprises a date palm broth. In some instances, date palm broth can be prepared by soaking date paste in water or from mixing dried date powder with water. For example, in some embodiments, date broth is prepared by soaking date 150 g of date paste in distilled water and heated at 60° C. for 3 h while mixing every 30 min. The resulting date slurry may then be mixed for another 1 h at room temperature and filtered through a cheesecloth to obtain the date broth. In another embodiment, 150 g of dried date powder may be combined with 1 L of distilled water and filtered through cheesecloth to obtain the date broth. In some embodiments, the date broth is the only source on nutrients in the growth medium.

In some embodiments the growth medium is supplemented with one or more of the following: L-cysteine hydrochloride, di-sodium phosphate, ammonium citrate, sodium acetate, calcium chloride, potassium phosphate, magnesium sulfate, manganese sulphate, arginine, Tween-80, and yeast extract. In some embodiments, the supplement solution may comprise L-Cysteine, HCl (1.5 g), disodium phosphate (2 g), ammonium citrate (2 g), sodium acetate (5 g), calcium chloride (0.3 g), MgSO₄·7H₂O (0.2 g), MnSO₄·5H₂O (0.05 g), arginine (0.5 g), yeast extract (20 g) and TWEEN 80 (3 mL) per 1 L of solution. In certain instances the date palm medium is an acidified medium. In some embodiments, the date palm medium comprises acetic acid. In some embodiments, the date palm medium comprises a 3:2 ratio of date broth:supplement solution.

In certain embodiments, the method comprises identifying one or more cultured microorganisms. Any methods known in the art may be used to identify the cultivated microorganisms. For example, the microorganisms may be cultured and pure colonies isolated to obtain microorganisms isolates. The isolates may be identified using 16S rRNA gene sequencing. The 16S ribosomal (r) RNA gene of bacteria encodes the small subunit of ribosomal RNA. The RNA sequences have been used extensively in the classification and identification of bacteria. The sequence of the 16S rRNA gene contains two regions: conserved and variable regions. The highly variable regions reflect differences between species. The comparison of the complete 16S rRNA gene sequence of an isolate against other sequences of close strains provides a rapid and accurate method to identify the bacterial strains. This method has been widely used to identify species by establishing taxonomic relationship between the strains with >97% sequence identity as the cutoff for distinguishing species and those with >95% sequence identity as the cutoff for distinguishing genera (Johnson et al. (2019) Evaluation of 16S rRNA gene sequencing for species and strain-level microbiome analysis. Nature Communications, 10, 5029).

In some embodiments, the methods further comprises determining the biological activity of one or more isolated microorganisms. Biological activities include, but are not limited to antibacterial and hemolytic activity. Microorganisms with antibacterial activity may be useful in a variety of applications. For example, microorganisms with antibacterial activities may be candidates to develop novel therapeutics. The biological activity of the microorganisms may be determined using any methods known in the art. For example, antibacterial activity may be determined using the cross streak method or well diffusion assays. Similarly, hemolytic activity can be determined using any methods known in the art. For example, in certain embodiment, hemolytic activity of selected bacterial isolates may be determined by streaking the isolates on TSA agar containing 5% sterile sheep blood and incubating at 30° C. or 37° C. for 48 hrs. After incubation, the hemolytic activity may be evaluated based on lysis of red blood cells in the medium surrounding the colonies. The clear zones around the colonies (0-hemolysis) indicate true hemolysis, while no zones around the colonies (7-hemolysis) indicate a lack of hemolytic activity.

Compositions Derived from the Characterization of Environmental Samples

In another aspect, disclosed herein is a composition comprising a consortium of distinct microorganisms. In some embodiments, microorganism formulations may be developed based off the biological activity of the consortium of microorganisms. In some embodiments, the microorganism-based formulations are suitable for agricultural and biotechnological purposes. For example, soil microorganism-based formulations may be used for a wide range of environmental bioremediation processes. Additionally and/or alternatively, organisms with probiotic properties, especially spore forming bacteria, can be used to develop a wide range of novel probiotic formulations. Additionally and/or alternatively, the composition may have therapeutic properties.

In some instances the composition comprises a consortium of distinct (i.e., different strains) microorganisms, wherein the consortium is at least 2 distinct microorganisms, optionally at least 5 distinct microorganisms, optionally at least 10 distinct microorganisms, optionally at least 20 distinct microorganisms, or optionally at least 50 distinct microorganisms.

In certain embodiments, the consortium of microorganisms comprises at least one microorganism characterized according to the methods described herein. In some embodiments, the microorganisms comprising the composition are selected based off their biological activity. Thus, in some embodiments, consortium of microorganisms comprises at least one microorganism characterized as having antibacterial activity. In some instances, the consortium of microorganisms comprises microorganisms without hemolytic activity.

In some embodiments, the formulations are stable at room temperature. In certain embodiments, the formulations have a shelf-life of at least 3, 6, 12, 18, 24, 36, 48, 54, 60, 66, 72, 78, 84, or 90 months at room temperature.

### EXAMPLES

The following examples describe compositions and methods for cultivating and characterizing environmental microorganisms.

### Example 1. Formulation of Growth Media Using Date Palm Fruits

An optimal growth medium contains all the nutrients for cultivation of bacteria, including a carbon source, nitrogen source, vitamins, minerals and trace elements. In a typical culture medium, these compounds are provided by several sources from both plant and animal ingredients. In some embodiments, the culture medium developed comprises a liquid broth prepared with date palm fruits. Date palm fruit, which is rich in sugar, amino acids and minerals (Table 1) was used as the base medium to enrich bacteria present in the formulation. In one example date broth alone was used for the cultivation of bacteria, while in another embodiment date broth supplemented with additional nutrients was used to enhance growth of fastidious organisms.

Date broth was prepared from either date paste or date powder (commercially available from LIWA, United Arab Emirates). The chemical compositions of the utilized date pastes and dried date powders are provided in Table 1. The date paste (150 g/L) was soaked in distilled water and heated at 60° C. for 3 h while mixing every 30 min. The resulting slurry was mixed for another 1 h at room temperature and filtered through a cheesecloth to obtain the date broth, which was autoclaved for 15 min before inoculation with the formulation. To prepare date broth using dried date powder, 150 g of the powder was mixed with 1000 mL of water, and the mixture was filtered through a cheesecloth after storing overnight at 4° C.

TABLE 1
Chemical Composition of Date Paste and Dried Date Powder

| | Chemical Composition of Dates | | | | |
|---|---|---|---|---|---|
| | | Al Ameen | | LIWA | |
| Component | Units | Paste | Powder | paste | powder |
| Ash | % | 1.80 | 1.94 | 1.79 | 2.58 |
| Calcium | mg/100 g | 60.10 | 86.00 | 53.70 | 115.00 |
| Iron | mg/100 g | 1.82 | 2.28 | 0.80 | 2.23 |
| Magnesium | mg/100 g | 58.50 | 78.30 | 62.40 | 94.30 |
| Manganeese | mg/100 g | 0.32 | 0.50 | 0.34 | 0.63 |
| Phosphorus | ppm | 573.00 | 766.00 | 526.00 | 861.00 |
| Potassium | mg/100 g | 738.00 | 879.00 | 793.00 | 1160.00 |
| Selenium | ppm | <0.10 | <0.10 | <0.10 | <0.10 |
| Sodium | mg/100 g | 4.40 | 4.76 | 6.18 | 7.43 |
| Zinc | mg/100 g | 0.35 | 1.79 | <0.5 | 1.40 |
| Moisture | % | 8.79 | 4.10 | 12.04 | 1.79 |
| Fat | % | 0.14 | 0.57 | 0.41 | 0.43 |
| Protein | % | 2.33 | 3.19 | 2.15 | 3.28 |
| Total sugar | % | 71.50 | 69.70 | 69.70 | 74.20 |
| Fructose | % | 34.80 | 7.02 | 33.00 | 8.11 |
| Glucose | % | 36.70 | 10.90 | 36.70 | 9.09 |
| Sucrose | % | <0.5 | 51.80 | <0.5 | 57.00 |
| Maltose | % | <0.5 | <0.5 | <0.5 | <0.5 |
| Lactose | % | <0.5 | <0.5 | <0.5 | <0.5 |
| Total dietary fiber | % | 7.00 | 16.55 | 8.00 | 15.70 |
| Insoluble | % | 6.63 | 15.06 | 7.54 | 13.60 |
| Soluble | % | 0.38 | 1.49 | 0.45 | 2.12 |

### Preparation of Supplemented Date Medium

Most fastidious bacteria require a good nitrogen source and some vitamins and minerals in abundance for optimal growth. To enhance the growth of a wide range of bacteria, an improved medium was formulated by adding several key ingredients/trace elements together with extra essential nutrients to the date broth. The supplement solution was prepared separately by adding the ingredients listed in Table 2. This chemically defined medium with date was prepared by combining the date broth (120 mL) with the supplement solution (80 mL). The date broth plus supplement mixture was sterilized at 121° C. for 15 min for cultivation experiments.

TABLE 2
Composition of Supplements Added to Date Medium

| | Amount |
|---|---|
| Ingredients | (g/L) |
| L-cysteine hydrochloride, | 1.5 |
| di-Sodium phosphate, | 2 |
| Ammonium citrate, | 2 |
| Sodium acetate, | 5 |
| Calcium chloride | 0.3 |
| Potassium phosphate, | 2 |
| Magnesium sulfate, | 0.2 |
| Manganese sulphate | 0.05 |
| Arginine, | 0.5 |
| Yeast extract | 20 |
| Tween-80 | 3 ml |

### Example 2. Cultivation of Microorganisms

### Determination of the Microbial Load in a Soil Microorganism-Based Formulation

In products containing a consortium of microorganisms, the number of living bacteria may change over time, and some may have a shorter shelf life and/or stability. The number of live bacteria present in the Gaia Flush formulation was determined by cultivating the formulation using BHI broth as the growth medium. Briefly, diluted Gaia Flush samples (1:10) in phosphate buffered saline (PBS) were added to BHI medium (100 mL), and incubated at 30° C. for 48 h. After incubation, the cultures were enumerated, and streaked onto BHI agar plates. The number of microorganisms enumerated in the sample was 5.8×10⁷ CFU/ml. The BHI agar plates showed considerable growth from all dilutions used confirming the presence of a significant number of live bacteria in the Gaia Flush formulation.

### Comparison of Different Growth Media for Cultivation of Bacteria Present in the Formulation

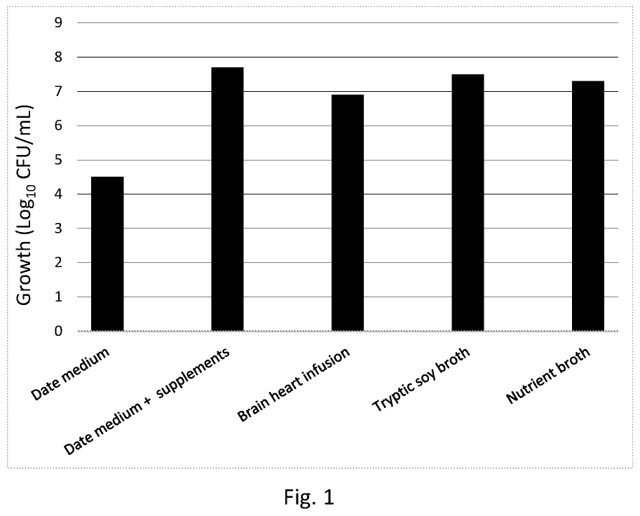

In order to compare and evaluate date media as an appropriate growth medium for culturing soil bacteria present in the Gaia Flush formulation, a comparative study was performed culturing the formulation in conventional culture media (BHI, TSB, nutrient broth) and date media (date broth and date broth supplemented with nutrient solution). Each medium (100 mL) was inoculated with I mL of diluted formulation (1/10) and incubated at 30° C. for 48 h. After incubation, the cultures were enumerated on respective agar plates (in the case of date media, BHI agar was used) using serial dilutions. As shown in FIG. 1, all media except for date medium without supplementation showed equal growth (approximately log 10⁷ CFU/mL). The date medium without supplementation produced lower growth (log 10⁴ CFU/mL). Subsequent growth experiments with the defined date medium indicated that incubation of the culture for an extended period (at least 5 days) resulted in higher growth. This experiment demonstrated that date medium supplemented with additional ingredients (Table 2) would function the same as commercially available culture media.

### Example 3. Isolation and Identification of Bacteria from a Soil-Based Formulation Cultivated in Date Palm Media

The Gaia Flush formulation was cultivated under four conditions: (i) date media without nutrient supplementation under aerobic conditions, (ii) date media with nutrient supplementation under aerobic conditions, (iii) pH-adjusted date media under aerobic conditions, and (iv) date media under anaerobic conditions. Pure colonies were isolated from the cultures and then stored at −80° C. with 20% glycerol.

(i) Cultivation of a Soil-Based Formulation in Date Media without Nutrient Supplementation Under Aerobic Conditions

The Gaia Flush formulation was cultured in date broth medium with no nutrient supplementations. The date medium was inoculated with diluted Gaia Flush samples in PBS (1/10 mL) and incubated for 30° C. for 48 h. After incubation, a loopful of the culture was streaked on agar media (BHI, TSA, and MRS agar plates). The plates were incubated at 30° C. for 48 hrs and the plates were examined daily for additional 5 days. The well isolated colonies were restreaked on the respective agar plates. The isolated colonies were then restreaked on another set of agar media (BHI and TSA) to obtain pure colonies.

(ii) Cultivation of a Soil-Based Formulation in Date Media with Nutrient Supplementation Under Aerobic Conditions

The date medium was inoculated with diluted Gaia Flush samples (1/10 ml) and incubated for 30° C. for 48 hrs. After incubation, a loopful of the culture was streaked on agar media (BHI, TSA, and MRS plates). The well separated colonies were restreaked on the agar media (BHI and TSA) and the plates were incubated at 30° C. overnight.

(iii) Cultivation of a Soil-Based Formulation in pH-Adjusted Date Media Under Aerobic Conditions

Most bacteria show optimal growth at neutral pH. However, some organisms like lactic acid bacteria grow well at a lower pH. The defined date medium was prepared by adding acetic acid, which lowered the pH to selectively enhance growth of Lactobacillus-related species. The date broth combined with supplement mixture was sterilized at 121° C. for 15 min. Acetic acid 1% was added to the sterilized broth to lower the pH to approximately 5. The diluted formulation (1/10) in PBS was added to the date broth and incubated at 30° C. for 48 h. Loopfuls of the culture were then streaked on MRS, TSA and BHI agar. The agar plates were incubated at 30° C. 1-2 days and well isolated colonies were restreaked on the respective agar plates.

### (iv) Cultivation of a Soil-Based Formulation in Date Media Under Anaerobic Conditions

As some soil bacteria prefer to grow in low oxygen milieus, this experiment was conducted to enrich anaerobic and facultatively anaerobic bacterial populations at 30° C. and 37° C. under anaerobic conditions. The diluted formulation (1/10) in PBS was added to the sterile date media and incubated at 30° C. or 37° C. for 2 days under anaerobic condition. Loopfuls of the culture were then inoculated on MRS, TSA, BHI, Reinforced Clostridial medium (RCM) and Wilkins-Chalgren agar. The agar plates were incubated for 2 days at 30° C. and/or 37° C. under anaerobic condition. Well isolated colonies were restreaked on respective agar plates.

### Example 4. Identification of Microorganisms Isolated from a Soil-Based Formulation

Sequencing of the 16S rRNA gene has been accepted as a standard for identification of bacterial strains. Pure colonies obtained from each agar plate were identified using 16S rRNA sequencing. In this study, sequencing of approximately 1600 bp 16S rRNA gene was achieved by Sanger sequencing. The sequences of the isolates were compared and the closest sequence to each isolate was searched using NCBI BLAST. Resulting hits were sorted by percent sequence identity along with the score. Isolates were assigned putative taxonomic identification based on the assumption that sequences of >97% identity represents the same species while those with >95% identity represents the same genus (Johnson et al. (2019) Evaluation of 16S rRNA gene sequencing for species and strain-level microbiome analysis. Nature Communications, 10, 5029).

All the bacterial strains isolated were Gram positive rods and the majority were spore formers. Date broth medium gave rise to an excellent growth of Bacillus spp., while the chemically defined buffered date medium under anaerobic conditions allowed the growth of Clostridium spp. and Sporolactobacillus spp. in abundance.

The major groups of identified bacteria were Bacilli, and Sporolactobacilli as shown in the Tables 3, 4, 5 and 6. Bacillus species included B. amyloliquefaciens, B. cereus, B. coagulans, B. lichenformis, B. megaterium, B. subtilis, B. thuringiensis and B. velezensis. Among them, both B. coagulans and B. subtilis are widely used as probiotics and the strains isolated in this study could be potential probiotics. 16S rRNA gene sequence alignments of those strains with known probiotic strains indicated these strains have nearly 98% similarity to known probiotics.

The 16S rRNA sequence was determined for Sporolactobacillus nakayame subsp. Racemicus (SEQ ID NO:1), Clostridium tyrobutyricum (SEQ ID NO:2), and Bacillus coagulans (SEQ ID NO:3).

SEQ ID NO: 1:
CTCAGGACGAACGCTGGCGGCGTGCNTAATACATGCAAGT
CGAGCGCATAGAAGGGAGCTTGCTCCCGGAAGTGAGCGGC
GGATGGGTGAGTAACACGTGGGTAACCTGCCTGTCAGACT
GGGATAACTGTGGGAAACCGCAGCTAATACCGGATAATCC
CCTGCACCGCATGGTGCAGGGCTGAAAGATGGTTTCGGCC
ATCACTGACAGATGGGCCCGCGGTGCATTAGTTAGTTGGC
GGGGTAACGGCCCACCAAGACAGCGATGCATAGCCGACCT
GAGAGGGTGATCGGCCACATTGGGACTGAGACACGGCCCA
AACTCCTACGGGAGGCAGCAGTAGGGAATCTTCCACAATG
GACGAAAGTCTGATGGAGCAACGCCGCGTGAGCGAAGAAG
GTTTTCGGATCGTAAAGCTCTGTTGCCGGAGAAGAACGTG
CGGGAGAGGAAATGCTCTTGCAGTGACGGTATCCGGCCAG
AAAGCCACGGCTAACTACGTGCCAGCAGCCGCGGTAATAC
GTAGGTGGCAAGCGTTGTCCGGAATTATTGGGCGTAAAGC
GCGCGCAGGCNNCTTCTTAAGTCTGATGTGAAATCTTGCG
GCTCAACCGCAAACGGTCATTGGAAACTGGGAAGCTTGAG
TACAGAAGAGGAGAGTAGAATTCCACGTGTAGCGGTGAAA
TGCGTAGATATGTGGAGGAATACCAGTGGCGAAGGCGGCT
CTCTGGTCTGTAACTGACGCTGAGGCGCGAAAGCGTGGGG
AGCAAACAGGATTAGATACCCTGGTAGTCCACGCCGTAAA
CGATGAATGCTAGGTGTTAGGGGGTCCAACCCTTAGTGCT
GAAGTTAACACATTAAGCATTCCGCCTGGGGAGTACGACC
GCAAGGTTGAAACTCAAAGGAATTGACGGGGGCCCGCACA
AGCAGTGGAGCATGTGGTTTAATTCGAAGCAACGCGAAGA
ACCTTACCAGGTCTTGACATCCTCTGACAAGTCTAGAGAT
AGGCCGTTCCCCTTCGGGGGACAGAGTGACAGGTGGTGCA
TGGTTGTCGTCAGCTCGTGTCGTGAGATGTTGGGTTAAGT
CCCGCAACGAGCGCAACCCTTGATCTTAGITGCCAGCATT
CAGTTGGGCACTCTAAGGTGACTGCCGGTGACAAACCGGA
GGAAGGTGGGGATGACGTCAAATCATCATGCCCCTTATGA
TCTGGGCTACACACGTGCTACAATGGGTGGTACAAAGGGC
AGCGAAACCGCGAGGTCGAGCTAATCCCATAAAGCCACCC
CCAGTTCGGATTGCAGGCTGCAACTCGCCTGCATGAAGCC
GGAATTGCTAGTAATCGCGGATCAGCATGCCGCGGTGAAT
CCGTTCCCGGGCCTTGTACACACCGCCCGTCACACCACGA
GAGTTTGTAACACCCGAAGTCGGTGCGAGAACCTTTATGG
ACTCAGCCGCCGAANGTGGGACAAATGATTGGGGTGAAGT
CGT
SEQ ID NO: 2
GCTCAGGACGAACGCTGGNNGCGTGCCTAACACATGCAAG
TCGAGCGATGAAACCCCTTCGGGGGTGGATTAGCGGCGGA
CGGGTGAGTAACACGTGGGTAANNGCCTCAAAGTGGGGGA
TAGCCTTCCGAAAGGAAGANTAATACCGCNTNAAGCCAAG
TTTCACATGGAATTTGGATGAAAGGAGTAATTCGCTTTGA
GATGGACCCGCGGCGCATTAGTTAGTTGGTGGGGTAATGG
CCTACCAAGACAGCGATGCGTAGCCGACCTGAGAGGGTGA
TCGGCCACATTGGAACTGAGATACGGTCCAGACTCCTACG
GGAGGCAGCAGTGGGGAATATTGCACAATGGGCGAAAGCC
TGATGCAGCAACGCCGCGTGAGTGATGAAGGTCTTCGGAT
TGTAAAGCTCTGTCTTTTGGGACGATAATGACGGTACCAA
AGGAGGAAGCCACGGCTAACTACGTGCCAGCAGCCGCGGT
AATACGTAGGTGGCGAGCGTTGTCCGGATTTACTGGGCGT
AAAGGGTGCGTAGGCGGATGTTTAAGTGAGATGTGAAATA
CCCGGGCTTAACTTGGGTGCTGCATTTCAAACTGGATATC
TAGAGTGCAGGAGAGGAGAATGGAATTCCTAGTGTAGCGG
TGAAATGCGTAGAGATTAGGAAGAACACCAGTGGCGAAGG
CGATTCTCTGGACTGTAACTGACGCTGAGGCACGAAAGCG
TGGGTAGCAAACAGGATTAGATACCCTGGTAGTCCACGCC
GTAAACGATGAGTACTAGGTGTAGGAGGTATCGACCCCTT
CTGTGCCGCAGTAAACACATTAAGTACTCCGCCTGGGAAG
TACGATCGCAAGATTAAAACTCAAAGGAATTGACGGGGGC
CCGCACAAGCAGCGGAGCATGTGGTTTAATTCGAAGCAAC
GCGAAGAACCTTACCTGGACTTGACATCCCCTGAATAACC
TAGAGATAGGCGAAGCCCTTCGGGGCAGGGAGACAGGTGG
TGCATGGTTGTCGTCAGCTCGTGTCGTGAGATGTTAGGTT
AAGTCCTGCAACGAGCGCAACCCTTATTGTTAGTTGCTAA
CATTCAGTTGAGCACTCTAACGAGACTGCCGCGGTTAACG
CGGAGGAAGGTGGGGATGACGTCAATCATCATGCCCCCTT
ATGTCCAGGGCAACACACGTGCTACAATGGGCANAACAAA
GAGAAGCAATACCGCGAGGTGGAGCCAANCTCAAAAACTG
CTCNCAGTTCGGATTGCAGGCTGAAACTCGCCTGCATGAN
CTGGAGTTGCTAGTAATCGCGAATCANATGTCNCGNGAAT
ACGTTCCCGGGCCTTGTACACACCGCCCGTCCACCATGAA
ACTGGCACACCCGAAGTCCGTANNCTAACGTAANNGACGC
GNCCNANGTGGGGTTAGNGATNGGGGTNAANTCNTANCAA
GTAACCGNAA
SEQ ID NO: 3
GGNNNNNNGCTCAGGACGAACGCNGGCGGCGTGCCTAATA
CATGCAAGTCGTGCGGACCTTTTAAAAGCTTGCTTTTAAA
AGGTTAGCGGCGGACGGGTGAGTAACACGTGGGCAACCTG
CCTGTAAGATCGGGATAACGCCGGGAAACCGGGGCTAATA
CCGGATAGTTTTTTCCTCCGCATGGAGGAAAAAGGAAAGA
CGGCTTNGGCTGTCACTTACAGATGGGCCCGCGGCGCATT
AGCTAGTTGGTGGGGTAACGGCTCACCAAGGCAACGATGC
GTAGCCGACCTGAGAGGGTGATCGGCCACATTGGGACTGA
GACACGGCCCAAACTCCTACGGGAGGCAGCAGTAGGGAAT
CTTCCGCAATGGACGAAAGTCTGACGGAGCAACGCCGCGT
GAGTGAAGAAGGCCTTCGGGTCGTAAAACTCTGTTGCCGG
GGAAGAACAAGTGCCGTTCGAACAGGGCGGCGCCTTGACG
GTACCCGGCCAGAAAGCCACGGCTAACTACGTGCCAGCAG
CCGCGGTAATACGTAGGTGGCAAGCGTTGTCCGGAATTAT
TGGGCGTAAAGCGCGCGCAGGCGGCTTCTTAAGTCTGATG
TGAAATCTTGCGGCTCAACCGCAAGCGGTCATTGGAAACT
GGGAGGCTTGAGTGCAGAAGAGGAGAGTGGAATTCCACGT
GTAGCGGTGAAATGCGTAGAGATGTGGAGGAACACCAGTG
GCGAAGGCGGCTCTCTGGTCTGTAACTGACGCTGAGGCGC
GAAAGCGTGGGGAGCAAACAGGATTAGATACCCTGGTAGT
CCACGCCGTAAACGATGAGTGCTAAGTGTTAGAGGGTTTC
CGCCCTTTAGTGCTGCAGCTAACGCATTAAGCACTCCGCC
TGGGGAGTACGGCCGCAAGGCTGAAACTCAAAGGAATTGA
CGGGGGCCCGCACAAGCGGTGGAGCATGTGGTTTAATTCG
AAGCAACGCGAAGAACCTTACCAGGTCTTGACATCCTCTG
ACCTCCCTGGAGACAGGGCCTTCCCCTTCGGGGGACAGAG
TGACAGGTGGTGCATGGTTGTCGTCAGCTCGTGTCGTGAG
ATGTTGGGTTAAGTCCCGCAACGAGCGCAACCCTTGACCT
TAGTTGCCAGCATTNAGTTGGGCACTCTAAGGTGACTGCC
GGTGACAAACCGGAGGAAGGTGGGGATGACGTCAAATCAT
CATGCCCCTTATGACCTGGGCTACACACGTGCTACAATGG
ATGGTACAAAGGGCTGCGAGACCGCGAGGTTAAGCCAATC
CCAGAAAACCATTCCCAGTTCGGATTGCAGGCTGCAACCC
GCCTGCATGAAGCCGGAATCGCTAGTAATCGCGGATCAGC
ATGCCGCGGTGAATACGTTCCCGGGCCTTGTACACACCGC
CCGTCACACCACGAGAGTTTGTAACACCCGAAGTCGGTGA
GGTANCCTTTACGGAGCCAGCCGCCGAANNGGGACAGANG
ATNGGGGTGAAGTCGTACAGGG

Sporolactobacillus spp., which are close relatives of lactobacilli are an important group of spore-forming bacteria. Sporolactobacillus species have been considered as potential probiotics (Johnson et al. (2019) Evaluation of 16S rRNA gene sequencing for species and strain-level microbiome analysis. Nature Communications, 10, 5029) and with their ability to sporulate, this group of bacteria makes this genus heat-resistant compared to other lactic acid bacteria.

The identified Clostridium strains are common soil bacteria, and Clostridium tyrobutyricum and Clostridium butyricum are known to produce large amounts of butyric acid. Microbacterium oxdyans, Paenibacillus aceti, and Lysinobacillus spp. were also present to a lesser extent in the cultures.

TABLE 3
Bacterial strains isolated from Gaia Flush cultured in date broth and
date broth with supplements (S) at 30° C. under aerobic conditions

| | | % | Strain |
|---|---|---|---|
| Growth Medium | Strain Name | Identity | ID |
| Date broth | Bacillus subtilis | 99% | BFAM 26 |
| Date broth | Bacillus subtilis | 99% | BFAM 27 |
| Date broth | Bacillus megaterium | 99% | BFAM 28 |
| Date broth | Bacillus amyloliquefaciens | 99% | BFAM 29 |
| Date broth | Bacillus velezensis | 99% | BFAM 30 |
| Date broth | Bacillus velezensis | 99% | BFAM 31 |
| Date broth | Bacillus velezensis | 98% | BFAM 32 |
| Date broth | Bacillus velezensis | 99% | BFAM 33 |
| Date broth | Bacillus subtilis | 98% | BFAM 34 |
| Date broth | Bacillus licheniformis | 98% | BFAM 35 |
| Date broth | Microbacterium oxydans | 99% | BFAM 36 |
| Date broth | Microbacterium oxydans | 99% | BFAM 37 |
| Date broth | Bacillus subtilis | 99% | BFAM 38 |
| Date broth | Bacillus amyloliquefaciens | 98% | BFAM 39 |
| Date broth + S | Paenibacillus aceti | 99% | BFAM 40 |
| Date broth + S | Clostridium tertium | 97% | BFAM 41 |
| Date broth + S | Clostridium sartagoforme | 96% | BFAM 42 |
| Date broth + S | Lysinobacillus macroides | 99% | BFAM 43 |
| Date broth + S | Paenibacillus aceti | 99% | BFAM 44 |
| Date broth + S | Bacillus licheniformis | 98% | BFAM 45 |
| Date broth + S | Bacillus licheniformis | 99% | BFAM 46 |
| Date broth + S | Bacillus sp. | 97% | BFAM 47 |
| Date broth + S | Bacillus cereus | 92% | BFAM 48 |
| Date broth + S | Paenibacillus xylaniticus | 98% | BFAM 49 |

TABLE 4
Bacterial strains isolated from Gaia Flush cultured in date broth and
date broth with supplements (S) at 37° C. under aerobic conditions

| Culture | | % | Strain |
|---|---|---|---|
| medium | Strain Name | Identity | ID |
| Date + S | Sporolactobacillus nakayamae | 99% | BFAM 50 |
| Date + S | Sporolactobacillus terrae | 99% | BFAM 51 |
| Date + S | Sporolactobacillus nakayamae | 99% | BFAM 52 |
| Date + S | Sporolactobacillus nakayamae | 98% | BFAM 53 |
| Date + S | Sporolactobacillus nakayamae | 98% | BFAM 54 |
| Date + S | Bacillus thuringiensis | 92% | BFAM 55 |
| Date + S | Bacillus thuringiensis | 94% | BFAM 56 |
| Date + S | Bacillus pumilus | 94% | BFAM 57 |
| Date + S | Bacillus cereus | 90% | BFAM 58 |
| Date + S | Sporolactobacillus laevis | 92% | BFAM 59 |
| Date + S | Sporolactobacillus nakayamae | 99% | BFAM 60 |
| | subsp. racemicus | | |

TABLE 5
Bacterial Bacterial strains isolated from Gaia Flush
cultured in date broth and date broth with supplements
(S) and acidified (A) at 30° C. Anaerobic conditions

| | | % | Strain |
|---|---|---|---|
| Culture medium | Strain Name | Identity | ID |
| Date + S | Clostridium tyrobutyricum | 99% | BFAM 60 |
| Date + S | Clotridium butyricum | 99% | BFAM 61 |
| Date + S | Clotridium butyricum | 98% | BFAM 62 |
| Date + S | Clostridium tyrobutyricum | 92% | BFAM 63 |
| Date + S | Clostridium beijerinckii | 99% | BFAM 64 |
| Date + S | Clostridium tyrobutyricum | 99% | BFAM 65 |
| Date + S | Clostridium diolis | 99% | BFAM 66 |
| Date + S | Sporolactobacillus nakayamae | 99% | BFAM 67 |
| Date + S | Bacillus drentensis | 99% | BFAM 68 |
| Date + S | Clostridium tyrobutyricum | 99% | BFAM 69 |
| Date + S | Bacillus licheniformis | 99% | BFAM 70 |
| Date + S | Panebacillus odorifer | 98% | BFAM 71 |
| Date + S + A | Paenibacillus macerans | 91% | BFAM 72 |
| Date + S + A | Clostridium tyrobutyricum | 99% | BFAM 73 |
| Date + S + A | Clostridium aurantibutyricum | 98% | BFAM 74 |
| Date + S + A | Staphylococcus caprae | 99% | BFAM 75 |
| Date + S + A | Brevibacterium frigoritolerans | 98% | BFAM 76 |

TABLE 6
Bacterial strains isolated from Gaia Flush cultured
in date broth and date broth with supplements
(S) at 37° C. under anaerobic conditions

| Culture | | % | Strain |
|---|---|---|---|
| medium | Strain Name | Identity | ID |
| Date broth | Bacillus coagulans | 99% | BFAM 77 |
| Date broth | Bacillus coagulans | 98% | BFAM 78 |
| Date broth | Bacillus coagulans | 99% | BFAM 79 |
| Date broth | Bacillus coagulans | 99% | BFAM 80 |
| Date broth | Bacillus coagulans | 99% | BFAM 81 |
| Date broth | Bacillus coagulans | 99% | BFAM 82 |
| Date broth | Bacillus coagulans | 99% | BFAM 83 |
| Date broth | Bacillus coagulans | 99% | BFAM 84 |
| Date broth | Bacillus sp. | 99% | BFAM 85 |
| Date broth | Sporolactobacillus nakayamae subsp. | 99% | BFAM 86 |
| | racemicus | | |
| Date + S | Sporolactobacillus nakayamae subsp. | 98% | BFAM 87 |
| | racemicus | | |
| Date + S | Sporolactobacillus nakayamae subsp. | 97% | BFAM 88 |
| | racemicus | | |
| Date + S | Clostridium tyrobutyricum | 97% | BFAM 89 |
| Date + S | Sporolactobacillus nakayamae subsp. | 99% | BFAM 90 |
| | racemicus | | |
| Date + S | Sporolactobacillus nakayamae subsp. | 99% | BFAM 91 |
| | racemicus | | |
| Date + S | Clostridium tyrobutyricum | 99% | BFAM 92 |
| Date + S | Sporolactobacillus nakayamae subsp. | 99% | BFAM 93 |
| | racemicus | | |
| Date + S | Sporolactobacillus nakayamae subsp. | 99% | BFAM 94 |
| | racemicus | | |
| Date + S | Sporolactobacillus nakayamae subsp. | 99% | BFAM 95 |
| | racemicus | | |
| Date + S | Sporolactobacillus vineae | 99% | BFAM 96 |
| Date + S | Sporolactobacillus nakayamae subsp. | 99% | BFAM 97 |
| | racemicus | | |
| Date + S | Bacillus sp. | 99% | BFAM 98 |

### Example 5. Antibacterial Activity

### Antibacterial Activity—Cross Streak Method

The test strains (soil isolates) were prepared in appropriate growth media (BHI or TSB). Each strain was inoculated on an agar plate by a single streak in the center of the petri dish and incubated at 30° C./37° C. for 1-2 days to provide enough growth to produce the antibacterial substance(s), that diffuse into the agar. The pathogens (Bacillus cereus [ATCC 14579] and Staphylococcus aureus [ATCC 25923]) were then streaked perpendicular to the test strain. The pathogen streaks were marked from the start point to the end to ensure clear zones are only due to the antibacterial action.

### Antibacterial Activity—Well Diffusion Assay

Diluted pathogen cultures Bacillus cereus (ATCC 14579) and Staphylococcus aureus (ATCC 25923) (1/100 in PBS [approximately 0.5 McFarland turbidity; 1-2×10⁶ CFU/ml]) were spread evenly over the entire surface of the agar plates (BHI or TSA) using a sterile cotton swab. After allowing the plates to air-dry for approximately 10 min, wells (7 mm) were cut using a sterile pipette tip (1000 μL). The wells were then filled with 100-150 μL of the isolated bacterial cultures. Ampicillin (10 μg) was used as a positive control and PBS was added as negative control. The plates were incubated at 37° C. for 22-24 h. The clear zones of inhibition surrounding the wells were recorded.

TABLE 7
Antibacterial Properties of the tested strains

Antibacterial Activity

| Organism | Cross streak method | Well diffusion assay |
|---|---|---|
| Bacillus coagulans | + | + |
| Bacillus haynesii | + | + |
| Bacillus subtilis | + | + |
| Bacilus velezensis | + | + |

### Hemolytic Activity

The hemolytic activity of the selected bacterial isolates was determined by streaking the isolates on TSA agar containing 5% sterile sheep blood and incubating at 30° C. or 37° C. for 48 hrs. After incubation, the hemolytic activity was evaluated based on lysis of red blood cells in the medium surrounding the colonies. The clear zones around the colonies (0-hemolysis) were considered as true hemolysis, while no zones around the colonies (γ-hemolysis) were considered as lack of hemolysis. None of the tested isolates had hemolytic activity (TABLE 7).

TABLE 7
Hemolytic Activity of tested strains

| Organism | Hemolytic activity |
|---|---|
| Bacillus coagulans | − |
| Bacillus haynesii | − |
| Bacillus subtilis | − |
| Bacilus velezensis | − |

1. A method of using a plant-based growth medium to simultaneously cultivate a consortium of microorganisms, wherein the consortium comprises at least 2 distinct microorganisms from a sample.

2. The method of claim 1, wherein the growth medium comprises a portion of date palm fruit.

3. The method of claim 2, wherein the portion of date palm fruit is a date paste or a date powder.

4. The method of claim 3, wherein the growth medium comprises a nutrient supplement solution, wherein the ratio of date paste:supplement solution is 3:2.

5. The method of claim 1, wherein the growth medium is acidified.

6. (canceled)

7. The method of claim 1, wherein the sample is an environmental sample.

8. The method of claim 7, wherein the environmental sample is a soil-based formulation.

9. A method of characterizing an environmental sample comprising:
(i) obtaining an environmental sample;
(ii) culturing the sample using a plant-based growth medium;
(iii) identifying at least one microorganism in the sample; and
(iv) determining the biological activity of at least one microorganism identified in step (iii).

10. The method of claim 9, wherein the environmental sample is a soil-based formulation.

11. The method of claim 9, wherein the growth medium comprises a portion of date palm fruit.

12. The method of claim 11, wherein the portion of date palm fruit is a date paste or a date powder.

13. (canceled)

14. The method of claim 10, wherein the identified microorganisms in the soil-based formulation comprise at least one of Bacillus spp., Microbacterium spp. and Clostridium spp.

15. The method of claim 14, wherein the identified Bacillus spp. is a Bacillus velezensis.

16. (canceled)

17. The method of claim 10, wherein the identified microorganisms in the soil-based formulation comprise at least one of Bacillus spp and Sporolactobacillus spp.

18. The method of claim 17, wherein the identified Sporolactobacillus spp is a sporolactobacillus nakayamae.

19. (canceled)

20. The method of claim 10, wherein the identified microorganisms in the soil-based formulation comprises at least one of Clostridium spp, Bacillus spp and Paenibacillus spp.

21. The method of claim 20, wherein the identified Clostridium spp is a Clostridium butyricum.

22. (canceled)

23. The method of claim 10, wherein the identified microorganisms in the soil-based formulation comprise at least one of Bacillus spp, Sporolactobacillus spp. and Clostridium spp.

24. The method of claim 23, wherein the identified Bacillus spp. is a Bacillus coagulans.

25. (canceled)

26. The method of claim 9, wherein the biological activity of at least one identified microorganism shows antibacterial activity.

27. (canceled)

28. A composition comprising a consortium of the microorganisms identified and characterized by the steps of:
(i) obtaining an environmental sample;
(ii) culturing the sample using a plant-based growth medium;
(iii) identifying at least one microorganism in the sample; and
(iv) optionally determining the biological activity of at least one microorganism identified in step (iii), wherein the consortium comprises i-s at least 2 distinct microorganisms.
